(19) United States
(12) Patent Application Publication
(10) Pub. No.: US 2018/0356384 A1
(43) Pub. Date: Dec. 13, 2018

(54) METHOD AND SYSTEM FOR CENTRALIZED MANAGEMENT, MONITORING, AND CONTROLLED DELIVERY OF BIOLOGICAL COMPOUNDS TO FRUIT STORAGE ROOMS

(71) Applicant: AGROFRESH INC., Philadelphia, PA (US)

(72) Inventors: Dana FAUBION, Yakima, WA (US); Richard JACOBSON, Chalfont, PA (US); Arden Nathan REED, Wenatchee, WA (US)

(21) Appl. No.: 15/992,652

(22) Filed: May 30, 2018

Related U.S. Application Data
(60) Provisional application No. 62/516,906, filed on Jun. 8, 2017.

(51) Int. Cl. G01N 33/00; G01N 33/02

(52) U.S. Cl. CPC G01N 33/0073; G01N 33/0047; G01N 33/025; G01N 33/004; G01N 33/0039

(57) ABSTRACT

The present application relates to a method and system for managing, monitoring, and controlling delivery of one or more biological compounds to one or more fruit storage rooms from a centralized location.

### CROSS REFERENCE TO RELATED APPLICATIONS

This application claims the benefit under 35 U.S.C. § 119(e) of U.S. Provisional Application Ser. No. 62/516,906, filed on Jun. 8, 2017, the entire disclosure of which is hereby expressly incorporated herein by reference in its entirety.

### FIELD OF THE PRESENT APPLICATION

The present application relates to a method and system for managing, monitoring, and controlling delivery of one or more biological compounds to one or more fruit storage rooms from a centralized location.

### BACKGROUND

Common practice of the commercial fruit industry is to store fruit in a sealed room for up to twelve months post-harvest before the fruit is transported to a retail outlet. Often, these fruit storage rooms are not inspected at all during the storage period. Accordingly, decay and physiological problems that may occur within the stored fruit is not detected until the storage room is opened, after the fruit is ruined, and the owner and/or operator experiences dramatic financial and commercial losses.

Existing technologies detect and control the temperature and/or endogenous gases produced within a fruit storage room, such as carbon dioxide (CO₂) and oxygen (O₂). To date, there is no device or method that can help fruit storage owners and operators identify, monitor, and manage potential for decay and physiological problems of fruit stored in their storage facilities. Fruit growers that harvest large quantities and/or various types of one or more fruits also have increased risk of economic losses due to the potential for decay and/or physiological problems in stored fruit. If gone undetected, physiological decay, disorders, diseases, and/or other problems that develop in fruit storage rooms may be passed or spread among, between, and/or within storage rooms to infect more and more fruit.

The present disclosure describes a method and a system for predicting, prognosing, and/or preventing premature fruit maturity and other physiological problems in one or more fruit storage rooms, such as a plurality of fruit storage rooms. More specifically, the method of the present disclosure detects excessive ripening or senescence of fruit, such as apples and pears, by measuring and monitoring environmental conditions and biological compounds in the headspace of multiple fruit storage rooms. In addition, the method and system of the present disclosure initiate remedial actions to counter physiological changes, ethylene exposure, anaerobic conditions, and senescence detected in a plurality of fruit storage rooms. Importantly, the present method and system are capable of delivering a remediation compound or agent, such as a plant growth regulator compound (e.g., 1-MCP), from a central control location to one or more of the plurality of fruit storage rooms in order to prevent and/or remediate fruit damage.

The methods and systems of the present disclosure monitor and control the environment within fruit storage rooms utilizing a proprietary best practices database that enables owners to predict the storage life, viability, and marketability of stored fruit. The present method and system provides owners a tool to manage, monitor, prevent, and/or remediate decay of fruit crops during storage. The present methods and systems provide fruit storage owner and operators with a new stream of information enabling responsive decision-making and best storage practices. The present method and system help preserve the economic viability of post-harvest fruit crops.

### SUMMARY OF THE INVENTION

The present disclosure provides a method of improving and/or maintaining quality in a plurality of plants or plant parts. The method may comprise enclosing a plurality of plants or plant parts in one or more storage rooms. The one or more storage room of the present method may be a contained environment.

The method may also comprise detecting the levels of one or more biological compounds present in the one or more storage rooms. In addition, the method comprises correlating the detected level of the one or more biological compounds in the one or more storage rooms with the quality of the plurality of plants or plant parts. Further, the method comprises simultaneously applying a remediation agent to the one or more storage rooms. Finally, the method comprises improving the quality of the plurality of plants or plant parts comprised in the one or more storage rooms.

The plurality of plants or plant parts of the present method may comprise fruit. For example, the fruit of the present method may be selected from the group consisting of apples, pears, avocados, bananas, carambolas, cherries, oranges, lemons, limes, mandarins, grapefruits, coconuts, figs, grapes, guavas, kiwifruits, mangos, nectarines, cantaloupes, muskmelons, watermelons, olives, papayas, passionfruits, peaches, persimmons, pineapples, plums, pomegranates, strawberries, blackberries, blueberries, and raspberries. More specifically, the fruit of the present method may be selected from the group consisting of apples and pears.

The biological compounds of the present method may be ozone, carbon dioxide, oxygen, nitrogen, and cyclopropene. The one or more remediation agents of the present method is cyclopropene. The cyclopropene compound of the present method is 1-methylcyclopropene.

The quality of the plurality of plants and plant parts comprised in the one or more storage rooms may be assessed by fruit immaturity, proper maturity, or over maturity. The one or more storage rooms of the present method is a contained environment. The present method of further comprises improving the quality of the plurality of plants and plant parts.

The present disclosure is also directed to a system of maintaining quality in a plurality of plants or plant parts. The instant system comprises a centralized control station comprising a software interface, a sensor, a preconcentrator, a dosing module comprising one or more remediation agents, and one or more storage rooms comprising one or more biological compounds, wherein the centralized control station is connected to the sensor, the preconcentrator, the dosing module, and the one or more storage rooms by electrical connections or gas connections.

The plurality of plants or plant parts of the present system may comprise fruit. For example, the fruit of the present disclosure may be selected from the group consisting of apples, pears, avocados, bananas, carambolas, cherries, oranges, lemons, limes, mandarins, grapefruits, coconuts, figs, grapes, guavas, kiwifruits, mangos, nectarines, cantaloupes, muskmelons, watermelons, olives, papayas, passionfruits, peaches, persimmons, pineapples, plums, pomegranates, strawberries, blackberries, blueberries, and raspberries. More specifically, the fruit of the present system may be selected from the group consisting of apples and pears.

The one or more biological compounds of the present system may be ozone, carbon dioxide, oxygen, nitrogen, and cyclopropene. The one or more remediation agents of the present system is cyclopropene. The cyclopropene compound of the present system is 1-methylcyclopropene.

The quality of the plurality of plants and plant parts comprised in the one or more storage rooms may be assessed in the present system by fruit immaturity, proper maturity, or over maturity. The one or more storage rooms of the present system is a contained environment. The present system further comprises improving the quality of the plurality of plants and plant parts.

### BRIEF DESCRIPTION OF THE DRAWINGS

A brief description of the drawings is as follows.

FIG. 1 is a schematic of a centrally controlled molecular-based fruit storage management and delivery system comprising components selected from the group consisting of 1) a central control station comprising a software interface, 2) a biological compound sensor, 3) a compound preconcentrator, 4) a compound source or dosing module for a remediation agent, such 1-methylcyclopropene or 1-MCP, and 5) one or more fruit storage rooms or chambers.

FIG. 2 is a graph showing the correlation between the ethylene response in apples stored in a fruit storage chamber comprising 1-MCP and a control fruit storage chamber that did not comprise 1-MCP over a 24-hour period. This data demonstrates confirmation by the biofeedback of the present method and system that the 1-MCP remediation treatment was effective on the treated apples.

### DETAILED DESCRIPTION

The term “plant(s)” and “plant parts” include, but not limited to, whole plants, plant cells, and plant tissues, such as leaves, calli, stems, pods, roots, fruits, flowers, pollen, and seeds. A class of plants that may be used in the present invention is generally as broad as the class of higher and lower plants including, but not limited to, dicotyledonous plants, monocotyledonous plants, agronomic crops, and horticultural crops. Horticultural crops include, but are not limited to crops such as, vegetable crops and fruit crops.

More specifically, horticultural crops of the present disclosure include, but are not limited to, fruit selected from, but not limited to, almond, apple, avocado, banana, berries (including strawberry, blueberry, raspberry, blackberry, currents and other types of berries), carambola, cherry, citrus (including orange, lemon, lime, mandarin, grapefruit, and other citrus), coconut, fig, grape, guava, kiwifruit, mango, nectarine, melons (including cantaloupe, muskmelon, watermelon, and other melons), olive, papaya, passionfruit, peach, pear, persimmon, pineapple, plum, and pomegranate. In particular, fruits (e.g., grapes, apples, pears, persimmons, and bananas) and berries (e.g., strawberries, blackberries, blueberries, and raspberries) are plants encompassed by the present disclosure. However, it should be noted that any variety or cultivar of berries or fruits may be used in the present invention

A vegetable is selected from the group, which include, but not limited to, asparagus, beet (including sugar and fodder beet), bean, broccoli, cabbage, carrot, cassava, cauliflower, celery, cucumber, eggplant, garlic, gherkin, leafy greens (lettuce, kale, spinach, and other leafy greens), leek, lentil, mushroom, onion, peas, pepper (sweet, bell, and/or hot peppers), potato, pumpkin, sweet potato, snap bean, squash, tomato and turnip. Nursery plant or flower or flower part is selected from the group, which include, but not limited to, rose, carnation, geranium, gerbera, lily, orchid, or other cut-flowers or ornamental flowers, flower bulbs, shrub, deciduous or coniferous tree.

The terms “plant material” or “plant part” include, but are not limited to, leaves, stems, roots, flowers or flower parts, fruits, cuttings, cell or tissue cultures, or any other part or product of a plant.

The methods of the present disclosure are directed to protecting horticultural plants and plant parts, such as fruit and vegetable crops, from degrading, diminishing, and/or reducing quality issues or problems (e.g., ripeness, over ripening, and senescence) during storage. The methods of the present disclosure predict, measure, detect, prognose, prevent, and/or remediate physiological quality issues or problems in plant or plant parts, such as fruit. Consequently, the present method and system improve and/or maintain crop quality in fruit and vegetable crops stored in storage rooms.

The phrase “one or more” is used in the present disclosure to refer to a single object (i.e., one object) or more than one object (i.e., two or more objects). The terms “multiple” or the phrase “a plurality” are interchangeably used in the present disclosure to refer to any and all numbers or amounts of objects, (e.g., fruit) that is more than one. Although not limited by any specific number or amount, a “multiple” or “a plurality” of objects may comprise 2 or more, 3 or more, 5 or more, 10 or more, 25 or more, 100 or more, 1000 or more, 2000 or more, 5000 or more, 10,000 or more, 100,000 or more objects. While there is no numerical limit, a “multiple” or “a plurality” of objects may comprise about 2 to about 1,000,0000, about 2 to 500,000, about 2 to about 100,000, about 2 to about 50,000, about 2 to about 25,000, about 2 to about 10,000, about 2 to about 5000, about 2 to about 2000, about 2 to about 1000, about 2 to about 500, about 2 to about 250, about 2 to about 100, 5 about 2 to about 50, about 2 to about 25, about 2 to about 20, about 2 to about 15, about 2 to about 12, about 2 to about 10, and about 2 to about 5.

While the present disclosure may apply to any fruit that is stored in storage rooms, illustrative fruit of the present disclosure includes apples and pears. For example, almost 250 varieties of apples and almost 90 varieties of pears are encompassed by the present disclosure. While not limited to those described herein, a detailed list of exemplary apple and pear varieties or cultivars included in the present disclosure is shown below in Table 1.

TABLE 1
Apple and Pear Cultivars

| | | Common Name |
|---|---|---|
| # | Common Name of Apple Cultivars | of Pear Cultivars |
| 1 | Adams Pearmain | Abate Fetel |
| 2 | Aia Ilu | Alexander Lucas |
| 3 | Airlie/Newell-Kimzey Red Flesh | Ambrosia |
| 4 | Akane | Ayers |
| 5 | Alkmene | Bambinella |
| 6 | Allington Pippin | Bartlett |
| 7 | Ambrosia | Black Worcester |
| 8 | Anna | Blake |
| 9 | Annurca | Blanquilla |
| 10 | Antonovka | Bon Rouge |
| 11 | Apollo | Bosc Pear |
| 12 | Ariane | Beurre Hardy |
| 13 | Arkansas Black | Butirra Precoce Morettini |
| 14 | Arthur Turner | Carmen |
| 15 | Ashmead's Kernel | Cascade |
| 16 | Aurora Golden Gala | Catillac |
| 17 | Autumn Glory | Churchland Pear |
| 18 | Bailey | Clairgeau |
| 19 | Baldwin | Clapp |
| 20 | Ballyfatten | Clara Frijs |
| 21 | Bardsey Island Apple | Concorde |
| 22 | Beacon | Conference |
| 23 | Beauty of Bath | Corella |
| 24 | Belle de Boskoop | Coscia |
| 25 | Ben Davis | D'Anjou |
| 26 | Beverly Hills | Dessertnaja |
| 27 | Birgit Bonnier | Don Guindo |
| 28 | Bismarck | Doyenné du Comice |
| 29 | Blenheim Orange | Dr. Jules Guyot |
| 30 | Bloody Ploughman | Earlibrite |
| 31 | Bottle Greening | Elektra |
| 32 | Braeburn | Flemish Beauty |
| 33 | Bramley (Bramley's Seedling) | Forelle |
| 34 | Bravo de Esmolfe | General Leclerc |
| 35 | Breedon Pippin | Gerburg |
| 36 | Brina | Giffard |
| 37 | Byfleet Seedling | Glou Morceau |
| 38 | Calville Blanc d'hiver | Gorham |
| 39 | Cameo | Harobig |
| 40 | Carolina Red June | Harovin Sundown |
| 41 | Carroll | Harrow Crisp |
| 42 | Carter's Blue | Harrow Delight |
| 43 | Catshead | Harrow Gold |
| 44 | Champion, Shampion or Sampion | Harrow Red |
| 45 | Charles Ross | Harrow Sweet |
| 46 | Chelmsford Wonder | Harvest Queen |
| 47 | Chiver's Delight | Hermann |
| 48 | Claygate Pearmain | Hortensia |
| 49 | Clivia | Huntington Pear |
| 50 | Cornish Gilliflower | Isolda |
| 51 | Cortland | Joséphine de Malines |
| 52 | Court Pendu Plat | Kieffer |
| 53 | Cox's Orange Pippin | Lategale |
| 54 | Crimson Gold | Laxton |
| 55 | Cripps Pink (‘Pink Lady’) | Le Conte |
| 56 | Crispin | Louise Bonne |
| 57 | Criterion | Luscious |
| 58 | D'Arcy Spice | Merton Pride |
| 59 | Delblush | Moonglow |
| 60 | Delcorf | Nashi |
| 61 | Delfloga | Kosui |
| 62 | Delflopion | Hosui |
| 63 | Delrouval | Nijisseiki |
| 64 | Deltana | Onward |
| 65 | Devonshire Quarreden | Orcas |
| 66 | Discovery | Orient |
| 67 | Dorsett Golden | Packham |
| 68 | Dougherty/Red Dougherty | Parsonage Pear |
| 69 | Duchess of Oldenburg | Pineapple |
| 70 | Dudley Winter | Red Bartlett |
| 71 | Dummellor's Seedling also known as | Rocha |
| | Dumelow's Seedling | |
| 72 | Early Victoria | Rosemarie |
| 73 | Edward VII | Seckel |
| 74 | Egle | Starkrimson |
| 75 | Egremont Russet | Stinking Bishop |
| 76 | Ein Shemer | Summer Beauty |
| 77 | Ellison's Orange | Summercrisp |
| 78 | Elstar | Sudduth |
| 79 | Emneth Early | Taylor |
| 80 | Empire | Tosca |
| 81 | Enterprise | Turandot |
| 82 | Envy | Uta |
| 83 | Epicure | Vicar of Winkfield |
| 84 | Esopus Spitzenburg | Virgouleuse |
| 85 | Falstaff | Warden |
| 86 | Fiesta | Williams |
| 87 | Fireside | Winter Nelis |
| 88 | Flamenco | |
| 89 | Florina | |
| 90 | Flower of Kent | |
| 91 | Fortune (Laxton's Fortune) | |
| 92 | Fuji | |
| 93 | Gala | |
| 94 | Garden Royal | |
| 95 | Gascoyne's Scarlet | |
| 96 | Geheimrat Dr. Oldenburg | |
| 97 | George Cave | |
| 98 | George Neal | |
| 99 | Ginger Gold | |
| 100 | Glockenapfel | |
| 101 | Gloster | |
| 102 | Golden Delicious | |
| 103 | Golden Noble | |
| 104 | Golden Orange | |
| 105 | Golden Russet | |
| 106 | Golden Spire | |
| 107 | Golden Supreme | |
| 108 | Goldspur | |
| 109 | Gradirose | |
| 110 | Gragg (aka Red Gragg, Winter Queen) | |
| 111 | Granny Smith | |
| 112 | Gravenstein | |
| 113 | Green Cheese | |
| 114 | Greensleeves | |
| 115 | Grenadier | |
| 116 | Grimes Golden | |
| 117 | Haralson | |
| 118 | Harrison Cider Apple | |
| 119 | Hawaii | |
| 120 | Herefordshire Russet | |
| 121 | Heyer 12 | |
| 122 | Honeycrisp | |
| 123 | Honeygold | |
| 124 | Howgate Wonder | |
| 125 | Idared | |
| 126 | Irish Peach | |
| 127 | James Grieve | |
| 128 | Jazz (Scifresh) | |
| 129 | Jonagold | |
| 130 | Jonathan | |
| 131 | Junaluska | |
| 132 | Junami | |
| 133 | Jupiter | |
| 134 | Kalmar Glasäpple | |
| 135 | Kanzi (Nicoter) | |
| 136 | Karmijn de Sonnaville | |
| 137 | Katy | |
| 138 | Kerry Pippin | |
| 139 | Kidd's Orange Red | |
| 140 | King | |
| 141 | King of the Pippins | |
| 142 | King Russet | |
| 143 | Knobbed Russet | |
| 144 | Lady Alice | |
| 145 | Lane's Prince Albert | |
| 146 | Laxton's Epicure | |
| 147 | Laxton's Fortune See ‘Fortune’ | |
| 148 | Laxton's Superb | |
| 149 | Liberty | |
| 150 | Limelight | |
| 151 | Liveland Raspberry apple | |
| 152 | Lodi | |
| 153 | Lord Derby | |
| 154 | Lord Lambourne | |
| 155 | Macoun | |
| 156 | Maiden's Blush | |
| 157 | Malinda | |
| 158 | Manks Codlin | |
| 159 | Mantet | |
| 160 | Margil | |
| 161 | May Queen | |
| 162 | McIntosh | |
| 163 | Melba | |
| 164 | Melrose | |
| 165 | Merton Charm | |
| 166 | Merton Worcester | |
| 167 | Miller's Seedling | |
| 168 | Mollie's Delicious | |
| 169 | Mother (American Mother) | |
| 170 | Muscadet de Dieppe | |
| 171 | Mutsu | |
| 172 | My Jewel | |
| 173 | Newell-Kimzey (Airlie Red Flesh) | |
| 174 | Newton Wonder | |
| 175 | Newtown Pippin (Albemarle Pippin) | |
| 176 | Nickajack | |
| 177 | Norfolk Royal | |
| 178 | Northern Spy | |
| 179 | Opal | |
| 180 | Orin | |
| 181 | Orleans Reinette | |
| 182 | Ozark Gold | |
| 183 | Pacific Rose | |
| 184 | Pam's Delight | |
| 185 | Paula Red | |
| 186 | Peasgood's Nonsuch | |
| 187 | Pink Pearl | |
| 188 | Pinova | |
| 189 | Pitmaston Pineapple | |
| 190 | Pixie | |
| 191 | Porter's | |
| 192 | Pott's Seedling | |
| 193 | Pound Sweet | |
| 194 | Prima | |
| 195 | Pristine | |
| 196 | Rajka | |
| 197 | Red Astrachan | |
| 198 | Red Delicious | |
| 199 | Red Prince | |
| 200 | Rev. W. Wilks | |
| 201 | Rhode Island Greening | |
| 202 | Ribston Pippin | |
| 203 | Rome Beauty | |
| 204 | Ros Picant | |
| 205 | Rosemary Russet | |
| 206 | Roxbury Russet | |
| 207 | Royal Gala See Gala | |
| 208 | Rubens (Civni) | |
| 209 | Santana | |
| 210 | Saturn | |
| 211 | Scrumptious | |
| 212 | Smokehouse | |
| 213 | Snow apple (Fameuse) | |
| 214 | Sonya | |
| 215 | Spartan | |
| 216 | Splendour/Splendor | |
| 217 | St. Edmund's Pippin | |
| 218 | Star of Devon | |
| 219 | Stark Earliest | |
| 220 | Stayman | |
| 221 | Streifling Herbst | |
| 222 | Sturmer Pippin | |
| 223 | Summerfree | |
| 224 | Sunset | |
| 225 | Suntan | |
| 226 | Sweet Sixteen | |
| 227 | SweeTango | |
| 228 | Teser | |
| 229 | Tolman Sweet | |
| 230 | Tom Putt | |
| 231 | Topaz | |
| 232 | Twenty Ounce | |
| 233 | Tydeman's Early Worcester | |
| 234 | Tydeman's Late Orange | |
| 235 | Wagener | |
| 236 | Warner's King | |
| 237 | Wealthy | |
| 238 | Westfield Seek-No-Further | |
| 239 | White Transparent | |
| 240 | Wijcik McIntosh | |
| 241 | Winesap | |
| 242 | Winston (Winter King) | |
| 243 | Wolf River | |
| 244 | Worcester Pearmain | |
| 245 | Wyken Pippin | |
| 246 | York Imperial | |
| 247 | Åkerö | |

### Compounds and Components of the Present Methods

The present disclosure is directed to methods and systems that manage, monitor, and prevent physiological problems and quality issues in fruit and/or vegetables stored in one or more storage rooms. In particular, the method and system described herein comprise detecting and/or measuring one or more components or compounds, such as biological compounds, in the headspace of a plurality of fruit storage rooms. When detected, the biological compounds or components serve as molecular signals for the methods and systems described herein.

Some biological compounds or components assessed by the present method and system may indicate physiological problems and/or quality issues simply by detection of their presence or absence at any concentration. Detection of other biological compounds or components may reach a particular level or threshold to indicate possible physiological problems or quality issues. In addition, the biological compound or component signal(s) may initiate remediation action by the instant method and or system to the fruit storage room in order to prevent, inhibit, and/or reduce damage to fruit stored within treated storage rooms.

The biological compound or biofeedback component of the present disclosure may comprise any chemical compound, molecule, and/or analyte that is associated, correlated, and/or predictive of a physiological disease or quality issue in a plant or plant part, such as fruit. For example, the biological compound may be any chemical compound, molecule, or analyte that is applied, produced, and/or generated during the natural ripening, growth, and/or storage process of fruit and vegetable crops. Illustrative compounds, molecules, or analytes of the present disclosure include, but are not limited to ethylene, ozone, carbon dioxide, oxygen, nitrogen, and cyclopropene. An exemplary cyclopropene compound of the present method is 1-methylcyclopropene or 1-MCP. In addition, exemplary compounds, molecules, or analytes of the present disclosure include other biological compounds.

Biofeedback components may also be detected and/or assessed by the present method and/or system. Illustrative biofeedback components of the present method and system include, but are not limited to carbon dioxide, heat/temperature, and oxygen consumption.

In particular, the present method and system may be used to measure biological compounds and/or biological feedback components that are byproducts of environmental conditions of the plants or plant parts (e.g., fruit) that are naturally produced in response to a biotic or abiotic stress. Thus, the present methods and systems may be used to manage, monitor, prevent, control, and remediate degradation or reduction of fruit quality. In addition, the methods and systems assist to restore health and vitality to plants, plant parts, including fruits and vegetables, being stored or transported in any volume of a contained environment, room, or fruit storage chamber for any length of time.

A single biological compound may be indicative of physiological problems and/or quality issues present in fruit and/or a fruit storage room. In addition, a combination or total of biological compounds may also be measured to indicate physiological problems and/or quality issues. For example, a group of biological compounds ranging from about 1 to about 20 compounds, from about 1 to about 15 compounds, from about 1 to about 10 compounds, from about 1 to about 5 compounds, from about 1 to about 3 compounds, from about 1 to about 4 compounds, and from about 1 to about 2 compounds.

When more than one biological compound or biofeedback component is measured, a total or combination of biological compounds or biofeedback components may have a particular index that indicates physiological problems and/or quality issues have been or will be detected by the methods and systems described herein. The index of biological compounds indicating physiological problems and/or quality issues may range from about 1 to millions based on the plant variety and conditions. With increased time in storage, repeat measurements of the biological compound index may change indicating a change in risk of onset of prevention or remediation of physiological or quality issues in fruit leading to the improvement or maintenance of quality of stored fruit.

More specifically, the presence of a biological compound or a biofeedback component of the instant disclosure may indicate a higher or lower probability of reduced quality (e.g., decay, ripening, advancement, or infection) of fruit. For example, the higher the concentration of a biological compound or a biofeedback component measured by the present method and system, the higher or lower may be the likelihood that a physiological problem and/or quality issue will develop in the tested storage room or the fruit comprised therein. In particular, the higher the measured concentration of any individual biological compound, combination of biological compounds, or total of biological compounds in the air, environment, or headspace of the one or more storage room of the present method or system, the higher the likelihood that a physiological problem and/or or quality issue has or will develop in the stored fruit. Conversely, higher concentrations of other measured biological compounds by the present method and system may indicate a lesser likelihood that a physiological problem and/or quality issue will develop in the tested storage room or the fruit comprised therein.

A measured concentration of the one or more biological compounds or biofeedback components in one or more fruit storage rooms may be compared to a different measurement to determine whether the total measured biological compounds or biofeedback components indicate physiological problems and/or quality issues (e.g., degradation, ripening, or advancement) of fruit. For example, biological compounds or biofeedback components measured in one or more fruit storage rooms may be compared to one or more comparator measurements or samples including, but not limited to: 1) the measured biological compounds or biofeedback components of the same storage room, for example, at an earlier time point and/or under different conditions, 2) the measured biological compounds or biofeedback components of one or more different storage rooms under the same or different conditions (see FIG. 2), or 3) a comparator sample or threshold measurement that is automatically or manually assessed, quantitated, and/or set in the instant method or system by a user.

Detection of some biological compounds or biofeedback components at any level (i.e., the presence or absence) may indicate physiological problems and/or quality issues within the stored fruit. For other biological compounds or biofeedback components that are measured, if the detected levels of the biological compounds or biofeedback components in the one or more storage rooms of interest are statistically significantly higher or lower than their respective comparator sample, measurements, or threshold, the measured level or concentration of the biological compound indicates that a physiological problem and/or quality issue has or will develop in the fruit stored in the one or more storage rooms being assessed. More specifically, if the ratio of biological compounds or biofeedback components measured in one or more chambers or storage rooms of interest as compared to one or more comparator samples or rooms is about 2:1 or greater or lower, about 1.75:1 or greater or lower, about 1.5:1 or greater or lower, about 1:25:1 or greater or lower, or about 1.1:1 or greater or lower, then the comparison ratio indicates that a physiological problem and/or quality issue has or will develop in the fruit stored in the one or more fruit storage rooms of interest.

The sample measurement detected for the one or more biological compounds or biofeedback components or molecules comprises, consists essentially of, and consists of a biological signal. The biological signal may comprise electronic signals, molecular signals, chemical signals, or biochemical signals, and/or some combination thereof. The biological signal may be delivered, received, analyzed, and/or translated by a computer comprising computer software that is controlled by a human user.

Once detected, the biological compound or biofeedback component signal from the tested fruit storage rooms is conveyed back to a centralized control center or station of the present method and system via a feedback mechanism within the method or system. Receipt, assessment, analyses, and confirmation of the biological compound or biofeedback component signal by the feedback loop mechanism of the present method or system then occurs.

At the central control station, the storage room, or some another location, the present method or system internally delivers, confirms, analyzes, compares, and monitors the measured level or concentration of the biological compound or biofeedback component signal to a comparator sample, measurement, or threshold. If the measured level or concentration of the one or more measured biological compound signals are outside of the acceptable threshold (e.g., higher/lower or present/absent), remediation action may be initiated, implemented, and/or completed by the instant method or system. Threshold samples or values to initiate, implement, and/or complete remediation action based on measured levels or concentrations of biological compound signals may be predetermined, entered, and/or incorporated into the present method or system by a human user. Threshold values may also be based on theoretical, predicted, or known measures, samples, or concentrations from the art.

In order to prevent or remediate any possible damage to fruit held within storage rooms, the methods and systems of the present disclosure may deliver and/or control delivery of a biological compound treatment or remediation agent or treatment. The biological treatment may originate from a compound source to plant or plant parts being held or stored within the fruit storage rooms and delivered in a dose-specific or time-dependent manner. One embodiment of the improvement, maintenance, prevention, and/or remediation action of the present method and system refers to the process of delivering or controlling delivery of a dose-specific amount of one or more biological remediation compounds or agents to affected fruit in one or more fruit storage rooms. Another embodiment of the prevention and/or remediation action of the present method and system refers to the process of delivering or controlling delivery of a dose-specific amount of one or more biological remediation compounds or agents to fruit in the one or more fruit storage rooms. In a further embodiment, the biological compounds are delivered to the fruit storage rooms over a specified time period in order to mitigate damage to fruit. Remediation action may be initiated, controlled, stopped, paused, and or restarted by the instant method and system manually, automatically, or semi-automatically.

One or more biological compounds comprised in a remediation compound treatment may be employed to prevent or remediate any damage to fruit. Remediation compounds and/or agents of the present method or system are biological compounds or chemical molecules that prevent, reverse, inhibit, modify, modulate, decrease, minimize, and/or reduce the effects of fruit damage, physiological problems, and/or quality issues that are or may be detected to be present, developing, or soon to develop in a fruit storage room. In particular, remediation treatments and compounds comprise, consist essentially of, or consist of biological or chemical molecules or combinations thereof that are efficacious against premature, enhanced, and/or advanced ripening that may be due to ethylene exposure of fruit, as described herein.

Illustrative remediation treatment compound agents of the present method and system comprise, consist essentially of, or consist of one or more cyclopropene compounds. The methods and systems of the present disclosure are directed to delivering one or more cyclopropene compounds in a dose-specific and/or time-dependent manner in order to remediate damage to horticultural plants and crops, such as fruit crops. An exemplary remediation compound agent of the present methods and systems comprise, consist essentially of, or consist of 1-Methylcyclopropene (1-MCP) compounds.

Exemplary embodiments of the remediation agents or compounds of the present disclosure comprise 1-Methylcyclopropene (1-MCP), which may encompass diastereomers and enantiomers of the illustrative compounds. Enantiomers are defined as one of a pair of molecular entities which are mirror images of each other and non-superimposable. Diastereomers or diastereoisomers are defined as stereoisomers other than enantiomers. Diastereomers or diastereoisomers are stereoisomers not related as mirror images. Diastereoisomers are characterized by differences in physical properties.

One exemplary embodiment of a 1-MCP compound of the present method is:

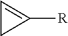

or an analog or derivative thereof. In an exemplary embodiment, R is methyl. 1-MCP may be used individually or as a mixture or combination with another compound or carrier. For example, the 1-MCP compound may also be used in combination with a carrier to form a 1-MCP remediation treatment. The 1-MCP active ingredient comprised in the treatment of the present disclosure comprises, consists of, or consists essentially of about 0.5% to about 50% active ingredient (e.g., 1-MCP) in the product. The 1-MCP remediation treatment provides protection to plants or crops from premature ripening when the treatment is administered, applied to, exposed to, and/or contacted with the plant or crops.

1-MCP may be used in the present method or system in any form, including, but not limited to, a liquid, a solid (e.g., a powder), a vapor, or a gaseous composition. In particular, the present method provides application of a 1-MCP compound as a spray, a mist, a gel, a thermal and non-thermal fog, a dip or a drench, or via sublimation, a vapor, or a gas. Additional examples of 1-MCP treatment administration include, but are not limited to, release from a sachet, a synthetic or natural film, a liner or other packaging materials, a gas-releasing generator, compressed or non-compressed gas cylinder, a droplet inside a box, or other similar methods. An exemplary embodiment of the remediation action of the present method or system comprises dose-specific delivery of 1-MCP gas or vapor to fruit storage rooms.

Carriers of the present disclosure are materials or compositions involved in carrying or transporting an active ingredient, compound, analog, or derivative from one location to another location. Carriers may be combined with one or more active 1-MCP compounds to form a 1-MCP remediation treatment. Treatment carriers of the present disclosure may comprise liquids, gases, oils, solutions, solvents, solids, diluents, encapsulating materials, or chemicals. For example, a liquid carrier of the present disclosure may comprise water, buffer, saline solution, a solvent, etc. Gas carriers of the present method may comprise nitrogen, oxygen, carbon dioxide, sulfur dioxide, and other gases.

The present methods and systems comprise, consist essentially of, or consist of a contained environment, wherein plants and fruit crops are stored and/or exposed to a remediation compound treatment. An illustrative contained environment is a storage chamber or a storage room. The storage chamber or storage room of the present method and system may be of any size that is large enough to hold or store plants or crops (e.g., fruit).

An exemplary fruit storage room or chamber of the present disclosure may be any contained environment, and may be sealable or non-sealable. Further, the storage room of the present method and systems may be air-tight, wherein compounds located in the environment within the storage room are not leaked to an environment outside of the storage room. Alternatively, the storage room or chamber may be less-than-air-tight, wherein insignificant amounts of leakage of the compounds located in the environment within the storage room are leaked to an environment outside of the storage room. In another embodiment, the storage room or chamber of the present disclosure must be sufficiently air-tight and/or impermeable such that the concentration of biological compounds that are measured, detected, and/or delivered to the fruit storage room are not substantially altered so as to further alter any measured biological compound, component, or response.

Any contained space that is used to hold plants, plant parts, or fruit crops may be used as a storage chamber or a storage room in the present method. For example, a storage room or chamber may be made of plastic, glass, wood, metal, stone/concrete, or any other semipermeable or impermeable construction materials used to store and/or transport plants or plant parts. Thus, the present method may be used post-harvest on plants or plant parts in greenhouse production, and during field packing, palletization, in-box, storage, and throughout the distribution network to predict degradation, problems, or issues in fruit crops.

For example, a fruit storage room or chamber includes, but is not limited to, a cold-storage room, controlled-atmosphere room, a marine container, an air container, a traincar or local vehicle, a transport truck or trailer, a box or a pallet-wrap, a greenhouse, a grain silo or similar. Further, other large industrial storage facilities are within the scope of the present disclosure of a fruit storage room.

A contained environment, storage chamber, or storage room of the present disclosure may be any contained volume of headspace from which a gas, vapor, or chemical cannot readily escape once it has been produced or introduced. Measurements of biological compounds in the atmosphere, air, or environment of the headspace of the chamber are conducted in the present method. While the chamber may be sealed or unsealed, a sealed chamber aids the accuracy of measurement of biological compounds in the chamber. The chamber of the present disclosure comprises a headspace (i.e., volume of capacity) that may be of any size or volume that is large enough to hold plants and plant parts to be analyzed.

An exemplary chamber may have a volume or headspace capacity of about 20 to about 2,000,000 pounds (lbs.) of fruit, from about 50 lbs. to about 1,750,000 lbs., from about 100 lbs. to about 1,500,000 lbs., from about 200 lbs. to about 1,250,000 lbs., from about 500 lbs. to about 1,100,000 lbs., from about 800 lbs. to about 1,000,000 lbs., from about 500 lbs. to about 2,000,000 lbs., and at about 1,500,000 lbs., 1,000,000 lbs. or about 2,000,000 lbs. of fruit.

The chamber may also have a port (e.g., a bulkhead septum port) for the introduction or capturing of a biological compound or component sample. The contained environment or storage chamber or room may also have an outlet to vent or release air, gas, or biological compounds in the storage chamber or storage room or to maintain atmospheric pressure.

The chamber comprises crops, such as fruit or vegetables, which are often contained in bins, boxes, recycled plastic containers and/or reusable plastic containers (RPC), or other types of containers. For example, a typical storage chamber may hold approximately 2000 bins or boxes of fruit. Each bin or box may be comprised of multiple layers. For example, exemplary bins or boxes of the present disclosure are comprised of about 2 to about 12 layers, from about 5 to about 15, from about 5 to about 12, from about 2 layers to about 15 layers, from about 2 layers to about 12 layers, about 2 layers, about 3 layers, about 4 layers, from about 5 layers, about 6 layers, about 7 layers, about 8 layers, about 9 layers, about 10 layers, about 11 layers, and about 12 layers.

### Methods and Systems of Administering Compounds to Fruit Storage Rooms

As described above, plants or crops, such as fruit crops, may be manually, automatically, or robotically placed in a storage room or chamber of the present method or system. The room may optionally be sealed. Fruit may be stored in the fruit storage rooms for 2, 4, 6, 8 or 10 weeks to 2, 4, 6, 8 10, 12 14, 16, 18, 24 months or more. During this storage time period the method and system of the present disclosure manage and monitor biological compounds released and/or produced by respiring fruit produce as well as other environmental biological compounds within the fruit storage room. When physiological problems or quality issues are detected, the method and system of the present disclosure engage in remediation action to minimize damage to stored fruit.

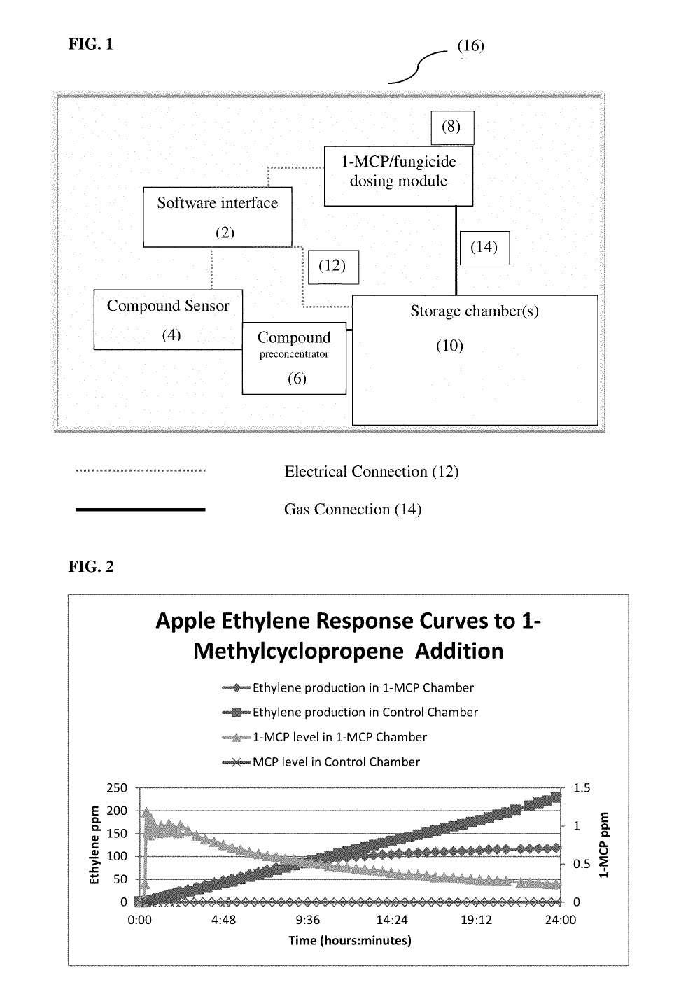

The present disclosure shows a schematic of an illustrative molecular-based fruit storage room management and delivery system that is centrally controlled (16; see FIG. 1). As shown in FIG. 1, the instant method and system (16) comprise components selected from the group consisting of a central control station (2) comprising a software interface (2), a biological compound sensor (4), a compound preconcentrator (6), a compound source or dosing module (8), and one or more fruit storage rooms or chambers (10).

While each component of the present method and system may be individually connected to the centralized control center (2), each component may also be connected to every other component, no other component, or specific other components of the method or system (16). Typically, the centralized control center (2) is connected to one or more, each and every, and/or all components of the present method and system (16) via physical, electric, electronic, and/or gaseous connections

Physical connections of the present inventions may comprise one or more tubes, hoses, conduits, or cables by which biological compounds, components, and detectors present in fruit storage rooms may travel, be measured, and remediation compounds and/or treatments may be delivered to fruit storage rooms. In particular, the tubes or tubing (14) of the present method and system (16) connects the central control center (2) to each of a plurality of fruit storage rooms (10) and/or delivers a dose-specific amount of the remediation treatment from a compound source or dosage module (8) to the fruit storage rooms (10). Alternatively, electric connections (12), such as cables and wires, send commands and signals to each component from the computer interface (2) at the control station (2).

For example, each of the fruit storage rooms or chambers (10) of the method or system (16) may also be connected to the centralized monitoring station (2) via tubes and/or tubing (14; not shown). In addition, the one or more fruit storage rooms (10) may be connected to each other via electrical (12) or gas tubing connections (14), such that commands, power, and/or biological compounds data, information, and signals may be shared between multiple fruit storage rooms of the present method.

The present fruit storage room management system (16) comprises a central control station (2), wherein the central control station (2) further comprises a software interface (2). The central control station (2) is the main hub and command center for the instant method and system (16). A human user interacts with and controls the computer software and interface (2) of the central control station (2) of the present system and method to interact, program, collect data, deliver commands and biological compounds, to a portion of or the entirety of the whole system (16). The central control station (2) is electrically connected (12) to the biological compound sensor (4), the compound source of dosing module (8), and the plurality of storage chambers (10).

The compound sensor (4) is a component of the present system (16) that is also connected to the compound preconcentrator (6) via electrical (12) and gas tubing (14) connections (not shown). Tubing (14) connecting the compound preconcentrator (6) to the fruit storage room (10) is used to remove and detect headspace air samples from the fruit storage room (10) for assessment for biological compound signals. Biological compounds in the fruit storage rooms (10) are concentrated in the compound preconcentrator component (6) before being measured by the compound sensor (4), which delivers the data results via electrical communication (6) to the software interface (2) of the central control station (2) for further analysis.

When levels and concentrations of detector compounds or biological compounds are analyzed and assessed by the software interface (2) of the present method or system (16) to indicate physiological problems and/or quality issues in fruit contained within the fruit storage rooms (10), the present system (16) engages in remediation action. For example, internal assessment of the measured biological compound by the software interface (2) to be above, below, or present/absent at a threshold that indicates physiological problems and/or quality issues and prompts the software interface of the present system (2) to relay messages to the compound source or dosing module (8) component via electric wiring (12).

The dosing module comprises the remediation agent (e.g., 1-MCP), which is released upon electronic command. In addition, the remediation action initiated by the dosing module (8) comprises delivery of a dose-specific and/or time-dependent treatment of one or more responsive compounds to the storage rooms (5) in which the detector compound or biological compound were originally detected. A compound source or dosing module (8) of the present disclosure may be any container, such as a tank or a drum, and may be sealable or non-sealable. Any container or drum that may contain, hold, and/or store a remediation compound treatment in its stable form (e.g., a solid, liquid, vapor or gaseous form) may be a compound source or dosing module (8) of the present disclosure.

Illustrative dosing modules of the instant remediation treatment or compound of the claimed method are tanks or drums that are able to stably store the remediation treatment. An exemplary compound source of the present system or method may be another fruit storage room. Another exemplary compound source (8) of the present disclosure includes, but is not limited to, a wagon, a transport truck cargo area, a cold-storage room, a marine container, an air container, a train car or local vehicle, a transport truck or trailer, a box, a pallet-wrap, a greenhouse, a grain silo, or similar.

The remediation agent of the present method is a plant growth inhibitor/regulator or a fungicide, an insecticide, or an herbicide. Exemplary remediation compounds provided by the compound source or dosing module (8) of the present method or system (16) comprise any known fungicide or plant growth regulator. An illustrative plant growth regulator or fungicide of the present method is 1-methylcyclopropene or 1-MCP. The 1-MCP remediation treatment may be applied to the fruit storage room or chamber via tubing (14) provided between the dosing module (8) and the one or more storage rooms (10) for an application time period. The duration of the application time period of the remediation treatment, e.g., 1-MCP, onto the plants or crops within fruit storage rooms depends on the number and type of fruit comprised therein.

For example, the plants may be exposed to the 1-MCP remediation treatment in the storage chamber for an initial time period ranging from about 5 seconds to about 30 minutes, and often times less than 5 seconds (i.e., 2 seconds, 3 seconds, or 4 seconds). However, the treatment time of 1-MCP to fruit within a fruit storage room may also comprise about 1 minute to about 5 days (120 hours), from about 2 minutes to about 4 days, from about 3 minutes to about 3 days, and from about 4 minutes to about 2 days, and from about 5 minutes to about 1 day after decay, disorder, or physiological problems are detected.

Measurement, assessment, and treatment of a plurality of fruit storage rooms of the present method and system may occur simultaneously, intermittently, or consecutively, and are centrally controlled form a single location (i.e., the software interface of the central control hub). In addition, different fruits and fruit storage rooms may be treated with the same or different remediation treatment compound(s), at treatment times, treatment temperatures, treatment pressures, etc. For example, the temperature of a fruit storage room or chamber of the present method or system during any treatment time period may remain at room temperature, which ranges from about 20° C. to about 23° C., or can be warmer.

In addition, the method and system described herein is capable of centrally controlling delivery, such as simultaneous delivery, of the remediation compound (e.g., 1-MCP) at a prescribed dose in order to efficiently and effectively treat various fruit storage rooms comprising fruits and vegetables. The sensor (4) and preconcentrator (6) of the system (16) then provide a feedback loop to specify the actual amount of 1-MCP delivered to the target storage room (10) so that remediation efficacy of fruit damage control by the prescribed treatment may be monitored and controlled.

If the initial remediation treatment is shown by subsequent monitoring and measurements of the present system to have lacked efficacy, plants and fruits in the one or more fruit storage rooms may be repeatedly treated by the same or different active ingredients, treatments, doses, times, and temperatures of treatment of the present method and/or system as necessary to prevent, mitigate, and/or reduce damage to stored fruit in fruit storage rooms. Thus, the present method and system provides an improvement over prior art fruit storage treatment methods that do not allow for the simultaneous, automated, and systematic assessment/detection and remediation treatment of fruit storage rooms as described herein.

The preceding description enables others skilled in the art to utilize the technology in various embodiments and with various modifications as are suited to the particular use contemplated. In accordance with the provisions of the patent statutes, the principles and modes of operation of this disclosure have been explained and illustrated in exemplary embodiments. Accordingly, the present invention is not limited to the particular embodiments described and/or exemplified herein.

It is intended that the scope of disclosure of the present technology be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

The scope of this disclosure should be determined, not only with reference to the above description, but should instead be determined with reference to the appended claims, along with the full scope of equivalents to which such claims are entitled. It is anticipated and intended that future developments will occur in the arts discussed herein, and that the disclosed compositions and methods will be incorporated into such future examples.

Furthermore, all terms used in the claims are intended to be given their broadest reasonable constructions and their ordinary meanings as understood by those skilled in the art unless an explicit indication to the contrary is made herein. In particular, use of the singular articles such as “a,” “the,” “said,” etc. should be read to recite one or more of the indicated elements unless a claim recites an explicit limitation to the contrary. It is intended that the following claims define the scope of the disclosure and that the technology within the scope of these claims and their equivalents be covered thereby. In sum, it should be understood that the disclosure is capable of modification and variation and is limited only by the following claims.

We claim:

1. A method of controlling the delivery of a remediation agent to a plurality of plants or plant parts comprising:
enclosing a plurality of plants or plant parts in one or more storage rooms,
detecting the levels of one or more biological compounds or biofeedback components present in the one or more storage rooms,
correlating the detected level of the one or more biological
compounds or biofeedback components in the one or more storage rooms with the quality of the plurality of plants or plant parts,
simultaneously initiating a dose of a remediation agent to the one or more storage rooms, and
controlling the delivery of the dose of the remediation agent to the
plurality of plants or plant parts comprised in the one or more storage rooms.

2. The method of claim 1, wherein the plurality of plants or plant parts comprise fruit.

3. The method of claim 1, wherein the one or more biological compounds are selected from the group consisting of ozone, carbon dioxide, oxygen, ethylene, nitrogen, and cyclopropene.

4. The method of claim 2, wherein the quality of the plurality of plants and plant parts is assessed by fruit immaturity, proper maturity, or over maturity.

5. The method of claim 1, wherein the one or more storage rooms is a contained environment.

6. The method of claim 2, wherein the fruit is selected from the group consisting of apples, pears, avocados, bananas, carambolas, cherries, oranges, lemons, limes, mandarins, grapefruits, coconuts, figs, grapes, guavas, kiwifruits, mangos, nectarines, cantaloupes, muskmelons, watermelons, olives, papayas, passionfruits, peaches, persimmons, pineapples, plums, pomegranates, strawberries, blackberries, blueberries, and raspberries.

7. The method of claim 6, wherein the fruit is selected from the group consisting of apples and pears.

8. The method of claim 1, wherein the one or more remediation agents is a cyclopropene compound.

9. The method of claim 8, wherein the cyclopropene compound is 1-methylcyclopropene.

10. The method of claim 1, wherein the one or more biofeedback components are selected from the group consisting of carbon dioxide, heat, and oxygen consumption.

11. A system for controlling the dose of a remediation agent to a plurality of plants or plant parts comprising:
a centralized control station comprising a software interface,
a sensor,
a preconcentrator,
a dosing module comprising one or more remediation agents,
and one or more storage rooms comprising one or more biological compounds or biofeedback components, wherein the centralized control station is connected to the sensor, the preconcentrator, the dosing module, and the one or more storage rooms by electrical connections or gas connections.

12. The system of claim 11, wherein the plurality of plants or plant parts comprise fruit.

13. The system of claim 11, wherein the one or more biological compounds are selected from the group consisting of ozone, carbon dioxide, oxygen, ethylene, nitrogen, and cyclopropene.

14. The system of claim 12, wherein the quality of the plurality of plants and plant parts are assessed by fruit immaturity, proper maturity, or over maturity.

15. The system of claim 11, wherein the one or more storage rooms is a contained environment.

16. The system of claim 12, wherein the fruit is selected from the group consisting of apples, pears, avocados, bananas, carambolas, cherries, oranges, lemons, limes, mandarins, grapefruits, coconuts, figs, grapes, guavas, kiwifruits, mangos, nectarines, cantaloupes, muskmelons, watermelons, olives, papayas, passionfruits, peaches, persimmons, pineapples, plums, pomegranates, strawberries, blackberries, blueberries, and raspberries.

17. The system of claim 16, wherein the fruit is selected from the group consisting of apples and pears.

18. The system of claim 11, wherein the one or more remediation agents is a cyclopropene compound.

19. The system of claim 18, wherein the cyclopropene compound is 1-methylcyclopropene.

20. The system of claim 11, wherein the one or more biofeedback components are selected from the group consisting of carbon dioxide, heat, and oxygen consumption.
